(19) United States
(12) Patent Application Publication
(10) Pub. No.: US 2006/0231093 A1
(43) Pub. Date: Oct. 19, 2006

(54) Pressurised inhalers

(76) Inventors: Simon Burge, Suffolk (GB); Warren Paul Isaacs, Huntingdon (GB); Simon James Smith, Hertford (GB); Jonathan David Tuckwell, Cambridge (GB)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/533,707

(22) PCT Filed: Nov. 4, 2003
(86) PCT No.: PCT/GB03/04751

§ 371 Date: Feb. 2, 2006

(30) Foreign Application Priority Data
Nov. 4, 2002 (GB) 0225655.0
Mar. 31, 2003 (GB) 0307378.0

(51) Int. Cl. A61M 11/00; A61M 16/00

(52) U.S. Cl. 128/203.15; 128/200.23

(57) ABSTRACT

A pressurised medicament canister has a valve which dispenses a metered dose upon being released from a depressed condition. An inhaler device adapted for use with the canister has a breath-actuated latch mechanism arranged in use to latch the canister in a depressed condition and further to release the latch in response to inhalation through the inhaler by a user. Additionally, a dose counter for counting the number of doses dispensed from the canister comprising a counter member having a helical toothed track which is incrementally advanced each time a dose is dispensed from the canister. Also a valve for the canister comprising a sliding seal delimiting the metering chamber and slidable relative to a nozzle member, said sliding seal being biased in use to reduce the volume of the metering chamber substantially to zero once the metering chamber has been vented to the atmosphere.

This invention relates to pressurised canisters for metered dose inhalers, valves for such canisters and to the inhalers per se.

Aerosol technology has been in existence for nearly a century using propellants or pressurised gas to deliver a fine liquid spray. An important development of this technology was a valve which delivered a fixed volume of fluid for each single actuation of the device. This is described in U.S. Pat. No. 22,723,055. It is fair to say that this development has revolutionised the drug delivery industry since fixed volumes of medication can be delivered using aerosol technology. This resulted in the advent of metered dose inhalers which are widely used today.

Metered dose inhalers have been used to treat asthma and other respiratory diseases for nearly 50 years and are currently the preferred method for delivering drugs to the lungs. However, there are a number of complications associated with the use of metered dose inhalers which limit their clinical effectiveness. Most significantly, there is a problem that standard inhaler devices require a degree of coordination on the part of the user that can make them difficult to use, particularly by certain groups of people such as the very young or very old. In particular, in order to use a metered dose inhaler correctly and successfully, the user must coordinate depressing the canister to dispense the dose with the first half of their inspiratory cycle. Failure to do this results in more limited quantities of the drug reaching the lungs than intended.

There have been many proposals in the prior art for overcoming this problem. The most elegant design of such a device is shown in WO 93/24167 and is embodied in the marketed “Easibreath” device. Other proposals can be seen in U.S. Pat. No. 5,511,540, WO 01/34231 and U.S. Pat. No. 5,347,998.

Whilst the devices described above can help to alleviate the problem, they all require a large number of components in order to provide a mechanism which is sufficiently powerful to provide the relatively large force (typically of the order of 30 Newtons) required to actuate the canister, yet which is sufficiently sensitive to be triggered by the user's breath. This large number of components makes such devices expensive and there is, therefore, a general reluctance to adopt them as standard drug delivery devices.

Another disadvantage in known metered dose inhalers is that users are advised to waste the first dose from the device when it has been unused for a significant period of time. The reason for this is that after each actuation, the return stroke of the nozzle causes a metering chamber within the canister to be refilled with the next dose. However, over a long period of time, there is a tendency for the active ingredient in the isolated dose to migrate out of the metering chamber thus reducing the net concentration of active ingredient and consequently reducing the therapeutic benefit of the dose held in the metering chamber.

Finally, the fact that a dose is always isolated in the metering chamber ready for dispensing in the next actuation, means that shaking the canister in order to obtain an even mix of propellant and active ingredient, as users are recommended to do, will be ineffective for the dose which will be next delivered.

It is the object of the present invention to alleviate the problems set out above. When viewed from a first aspect the invention provides a pressurised canister for a metered dose inhaler comprising a resiliently biased nozzle and arranged to dispense a metered dose of fluid from said nozzle upon releasing the nozzle from its depressed condition.

Thus it will be seen by those skilled in the art that the present invention represents a complete departure from the accepted assumption in the art that the dose is always delivered by pressing the nozzle. The Applicants now appreciate that there are several advantages arising from arranging to dispense the mixture of propellant and active ingredient upon the release stroke of the actuation of the nozzle rather than the initial depression stroke. One of the advantages of this arrangement is that it has been found that it is significantly easier for a human user to coordinate releasing the force required to actuate the nozzle of a canister with inhalation than it is to coordinate applying such force with inhalation. Thus, the user may provide the force to depress the nozzle into the canister without any coordination and then coordinate releasing the canister with inhalation.

More importantly, however, the reduced force required to release rather than to apply the actuation force means that a much more straightforward latch mechanism, operated directly by the user's in-breath, may be provided. The invention therefore also extends to an inhaler device comprising means for latching a canister in its depressed condition and means for releasing said latch upon inhalation by a user.

As well as the advantage of improving user coordination, in accordance with the invention, the Applicants have further realised that dispensing the dose in the second, release half of the actuation cycle makes it easy to arrange for the dose to be isolated during the same actuation cycle as it is dispensed. This has two main advantages. The first is that in normal use the dose to be dispensed will only be isolated for a very short period of time and there will therefore be insufficient time for the active ingredient to migrate out of it. This removes the need for a user to waste the first dose from the canister after it has not been used for a long period of time.

Secondly, the canister may be shaken prior to actuation, i.e. before the dose is isolated, which will result in a homogenous dose being dispensed. This reduces the risk of poor dose content uniformity.

When viewed from a further aspect, therefore, the present invention provides a pressurised canister for delivering a metered dose of fluid therefrom comprising a resiliently biased nozzle and arranged to isolate and deliver the same dose in a single actuation cycle. In other words, in each cycle of depressing and releasing the nozzle, a predetermined dose is isolated from the contents of the canister and dispensed from the nozzle.

It is envisaged that the dose may be isolated and dispensed during the same half of the actuation cycle. For example, the dose could be both isolated and dispensed on the depression stroke or, more preferably, isolated and dispensed on the release stroke. Most preferably, however, the dose is isolated during the depression stroke and dispensed during the release stroke. The advantages of dispensing during the release stroke for improving the ability to coordinate with breathing in are given above. The advantage of having the dose isolated in the other half of the cycle is that in general this arrangement minimises the length of stroke required.

It should be appreciated that although the present specification refers to isolating a dose, it should not be taken to imply that the isolated dose is sealed from the bulk of the canister's contents. It is sufficient that a predetermined volume of mixture is physically separated in some way from the remainder.

Many straightforward ways of implementing the arrangements set out above may be envisaged. In a preferred set of embodiments for example, the canister comprises a valve including a metering chamber and a hollow nozzle resiliently biased into a first position in which said nozzle is in fluid communication with the metering chamber, said nozzle being moveable against said resilient bias to a second position in which the metering chamber is in fluid communication with the interior of the canister. It will also be appreciated that the invention extends to a valve for a canister said valve comprising a metering chamber, an inlet for fluidly communicating with the interior of a canister and a hollow nozzle resiliently biased into a first position in which the nozzle is in fluid communication with the metering chamber, but moveable against said resilient bias into a second position in which the inlet is in fluid communication with the metering chamber.

Indeed, it will be appreciated that in general the invention extends to valves per se for pressurised canisters having the features of the canisters described hereinabove in accordance with the invention. When viewed from another aspect therefore the invention provides a valve for a pressurised canister, comprising a resiliently biased nozzle, the valve being arranged to dispense a metered dose of fluid from said nozzle upon releasing the nozzle from its depressed condition.

When viewed from a yet further aspect the invention provides a valve for a pressurised canister comprising a resiliently biased nozzle, said valve being arranged to isolate and deliver the same metered dose of fluid in a single actuation cycle.

The Applicants have devised a further improvement to the valves described hereinabove. When viewed from another aspect the present invention provides a pressurised canister for dispensing a metered dose of fluid therefrom having a valve comprising a sliding nozzle member biased towards a rest position but moveable against said bias to a priming position in which a metering chamber is defined within the valve such that when said nozzle member is released a metered dose is dispensed, the valve further comprising a sliding seal delimiting said metering chamber and slidable relative to the nozzle member, said sliding seal being biased in use to reduce the volume of the metering chamber substantially to zero once the metering chamber has been vented to the atmosphere via the nozzle member.

Thus it will be seen in accordance with this aspect of the invention that after the metered dose has been dispensed, a sliding seal reduces the volume of the metering chamber substantially to zero. This is beneficial since it ensures that the metering chamber is completely evacuated after the dispensing; thereby ensuring that a consistent dose is achieved each time. It also prevents the metering chamber being exposed to the atmosphere during storage which is sometimes perceived to be unhygienic. A further benefit is that the dose may be driven from the metering chamber at a substantially constant pressure which allows an optimal droplet size distribution to be maintained throughout the dispensing operation.

During the release stroke of the nozzle member the sliding seal moves past the communicating port between the metering chamber and the interior of the canister to seal the metering chamber before it is vented to the atmosphere. At this point the metering chamber contains an essentially incompressible volume of fluid. In some known designs this can lead to problems relating to the hydraulic lock which is thereby created. However, the ability of the seal in accordance with the invention to move independently of the nozzle member alleviates this problem since the nozzle member may continue under its restorative biasing force towards its rest position without reducing the volume of the metering chamber, with the sliding seal remaining in its position.

Once the nozzle member has moved to a position where the metering chamber is vented to the atmosphere through the nozzle member, the pressure in the metering chamber will drop and this may then cause the sliding seal again to slide so as to reduce the volume of the metering chamber substantially to zero.

Preferably the sliding seal is exposed to the pressure of the contents of the canister in order to apply at least some of the force required to move the seal. It is envisaged that the internal pressure of the canister could provide all of the required force. It is presently preferred however that a spring is provided within the valve to act on the sliding seal. Preferably the spring is arranged to act between the nozzle member and the sliding seal to give a biasing force on the sliding seal relative to the nozzle member. The force of such a spring will be less than the main restorative force, e.g. from a spring, acting on the nozzle member to bias it towards its rest position.

Where provided the spring may act directly on the seal. In some preferred embodiments however, an intermediate collar is provided to transmit force from the spring to the seal. Alternatively a hybrid comprising a collar with one or more resilient elements could be used.

The nozzle member may be biased towards its rest position by a spring, internal pressure within the canister or, preferably, a combination of the two.

Also disclosed herein is an inhaler device adapted for use with a pressurised canister having a valve which dispenses a metered dose therefrom upon being released from a depressed condition. In accordance with all aspects of the inventions set out below, it is preferred but not essential that the canister and/or valve is/are in accordance with the inventions and embodiments thereof described hereinabove.

When viewed from one aspect this invention provides a metered dose inhaler comprising means for receiving a pressurised medicament canister; and a breath-actuated latch mechanism arranged in use to latch said canister in a depressed condition and further to release said latch in response to inhalation through the inhaler by a user.

Thus it will be seen that in accordance with the invention set out above, an inhaler is provided in which the user's breath releases a latch holding the canister in its depressed condition to release a metered dose of medicament. As has been explained above, breath actuation offers significant benefits in co-ordinating inhalation with dispensing the dose.

The adaption of the inhaler in accordance with the invention to operate a canister which dispenses a metered dose upon being released (rather than as it is depressed, which is more common) allows a simple latch mechanism as was discussed previously. Many suitable mechanisms may be envisaged for providing the desired breath-actuated latch operation. In a particularly preferred embodiment however, the latch mechanism comprises a pivotally mounted latch arm operatively associated with a hinged flap arranged to rotate upon inhalation by a user. It will be appreciated that this gives the potential to provide a breath-operated dispensing mechanism, as in the preferred embodiments, with as few as two additional parts over a standard inhaler, which is to be contrasted with the complicated arrangements for breath-actuation in the prior art. Indeed arrangements may be envisaged in which just a single additional part is required.

Thus in accordance with at least preferred embodiments of the invention a hinged flap is provided which is placed so that air is drawn past it when the user inhales, causing the flap to move. In a particularly preferred embodiment the flap is provided so as to close an air inlet to the inhaler. This means that in its rest position the flap will close the inlet but upon inhalation by the user, air will be drawn into the device past the flap, thereby displacing it. The resultant movement may of course be used to release the latch. Conveniently for example the flap could be arranged across an air inlet aperture in a wall of the inhaler.

The flap may be restored to its rest position by any convenient means after it has been displaced, for example it may fall back under gravity, or a light restorative spring or some other resilient arrangement could be provided. Preferably however means are provided for positively restoring the flap. Preferably such a function is at least partly fulfilled through re-priming the latch mechanism, but additionally or alternatively an externally-operated actuator may be provided. Conveniently this actuator comprises or is operated by a cover for the mouthpiece of the inhaler which is arranged to restore or to help to restore the flap when the cover is closed over the mouthpiece.

In a further preferred feature which takes advantage of the mechanical force available from such an action, the external actuator, and thus preferably the mouthpiece cover, is arranged to apply a sealing force on the flap. This is beneficial in preventing the ingress of dust and dirt into the inhaler which might otherwise be in danger of being inhaled. It also locks the flap in place to prevent accidental actuation.

Such an arrangement is considered to be novel and inventive in its own right and thus when viewed from another aspect the invention provides a breath-actuated inhaler comprising a mouthpiece, a mouthpiece cover and an air inlet, the mouthpiece cover being arranged such that as it is brought over the mouthpiece it acts on a flap to hold the flap in a position where it closes the air inlet.

As previously, it is preferred that the cover acts to provide a sealing force on the flap.

During use the mouthpiece cover is moved away from the mouthpiece to allow access to it for the user's mouth. In accordance with a further preferred feature the inhaler is arranged such that in this open position, i.e. during use of the inhaler, the mouthpiece cover forms a guard over the air inlet to prevent inadvertent blockage of the air inlet, e.g. by the user's hand, during inhalation. Such inadvertent blockage of the air inlet can sometimes occur and causes problems with the proper inhalation of the required dose since entrainment of the medicament particles is impaired. It may also cause problems in operating a breath-actuated mechanism if there is an insufficient flow of air.

Such an arrangement is also considered to be novel and inventive in its own right and thus when viewed from a yet further aspect the invention provides an inhaler comprising a mouthpiece, a mouthpiece cover and an air inlet wherein the mouthpiece cover is movable from a first position in which it covers said mouthpiece to a second position in which it forms a guard over said air inlet to prevent blockage thereof in use.

The mouthpiece cover could be slidably or otherwise mounted. Preferably, it is pivotally mounted.

It has been discussed above that it is a preferred feature.of the invention that a flap is provided over an air inlet. Part of the reason why this is beneficial is that it prevents the ingress of dust, dirt etc. For a similar reason, it is a feature or preferred feature of all of the forgoing aspects of the invention that when in use a canister is inserted into the inhaler, the interior of the inhaler is substantially closed except for the mouthpiece and air inlet.

This contrasts with the conventional inhaler design in which the interior of the inhaler is generally open. For example, a passage of air is provided around the canister—indeed space around the canister is essential to admit air into the device.

The provision of a substantially sealed inhaler is beneficial from hygienic considerations and also helps to enhance the performance of the breath actuation mechanism.

It is desirable with metered dose inhalers to provide a means of keeping a count of the number of doses which have been dispensed from a particular canister so that ample warning is given of when it will be necessary to change the canister. Many users of inhalers carry two or more with them as a precaution against one running out or otherwise malfunctioning, but it is preferable not to have to rely on a back up inhaler routinely.

There have been many proposals for dose counters for inhalers in the past but these all have various drawbacks. A common problem encountered in designing mechanical counters for this application is that if the counter is based on a counting wheel, the relatively compact size of the inhaler means that it is impossible to provide enough graduations around the perimeter of the wheel to give anything but a very crude indication of the number of doses dispensed/remaining. This problem has tended to be overcome in the previous proposals either by using an electronic counter, which has the obvious disadvantages of cost and the need for a power source; or using multiple wheels which increases the cost and complexity. The result is that counters have yet to catch on widely in metered dose inhalers and there remains a need for a simple, cost-effective solution for providing a dose counter.

When viewed from one aspect another invention disclosed herein provides a metered dose inhaler for receiving a pressurised medicament canister and comprising a dose counter for counting the number of doses dispensed from said canister said dose counter comprising a counter member having a toothed track arranged substantially in a helix and means for incrementally advancing said counter member via said toothed track for each time a dose is dispensed from said canister.

Thus it will be appreciated that in accordance with the invention the helically arranged ratchet mechanism allows the indication of number of doses dispensed/remaining also to be arranged around a helix. This means that the number of counts displayed are not limited to those that will fit around the circumference of a counting wheel or the like; as many complete turns as desired may be used to accommodate the dose count indications. Consequently for example, digits which are large enough to be clearly visible may be used in any required number.

The use of a helical arrangement sacrifices the automatic resettability achieved with multiple wheels and electronic counters. However, the Applicants have appreciated that this is not a concern since resetting of the counter will normally always be associated with replacing or refilling the medicament canister.

The counter member could be driven by a simple ratchet mechanism. Preferably however an escapement-type mechanism is used in which a reciprocating motion from depressing and releasing the canister is translated into an incremental rotary motion of the counter member. This has been found to provide a reliable mechanism whilst minimising the number of parts required.

In particularly preferred embodiments the escapement mechanism comprises an escapement yoke comprising a pair of pawls which are arranged to engage with teeth on opposite sides of the toothed track when the canister is respectively depressed and released.

The counter drive mechanism, preferably an escapement-type mechanism, is preferably operatively associated with a canister latch mechanism, most preferably a canister latch mechanism as described hereinabove. Thus as the canister is primed and latched, one of the pawls engages one of the teeth on the toothed track to drive the counter half an increment and when the latch is released, the first pawl disengages and the second pawl engages to drive the counter through the rest of the incremental movement.

Certain preferred embodiments of the invention will now be described, by way of example only, with reference to the accompanying drawings in which:

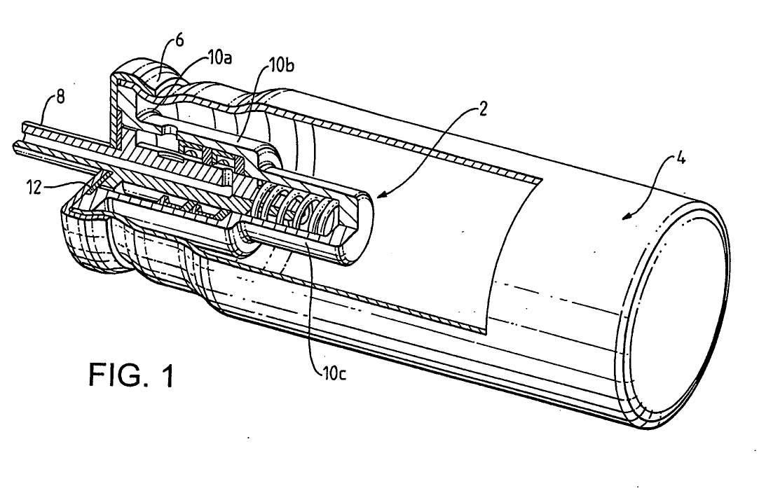

FIG. 1 is a partially cut-away perspective view of a pressurised canister and its valve in accordance with the invention;

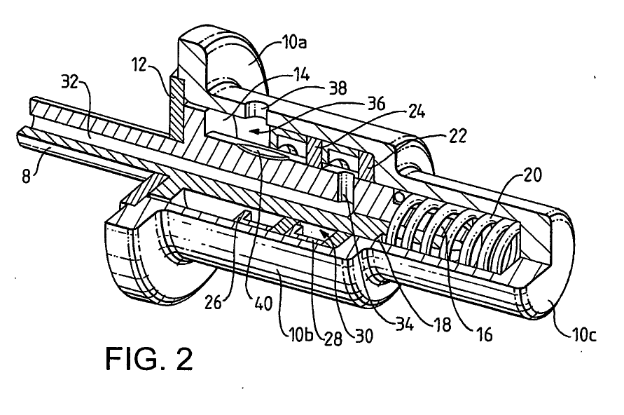

FIG. 2 is a close-up view of the valve of FIG. 1;

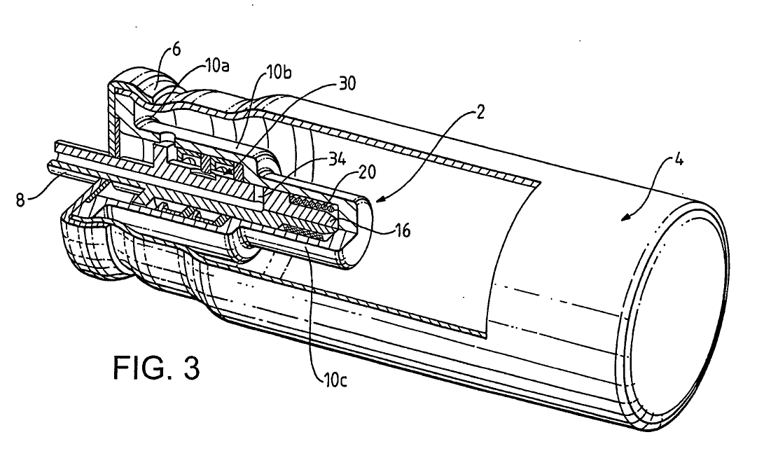

FIG. 3 is a view similar to FIG. 1 in which the nozzle is depressed;

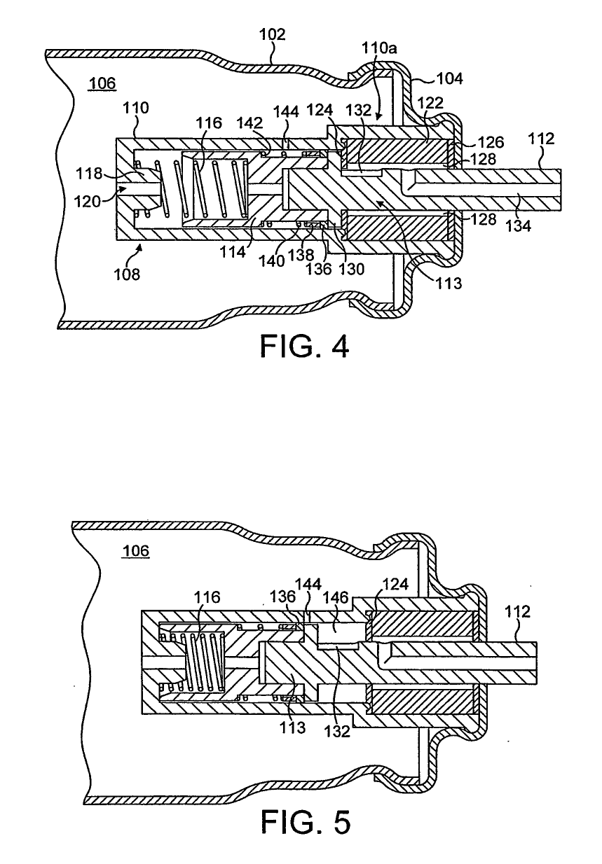

FIG. 4 is a sectional view through a valve in accordance with a second embodiment of the invention in a fully extended or rest state;

FIG. 5 is a sectional view similar to FIG. 4 showing the valve in a fully compressed or primed state;

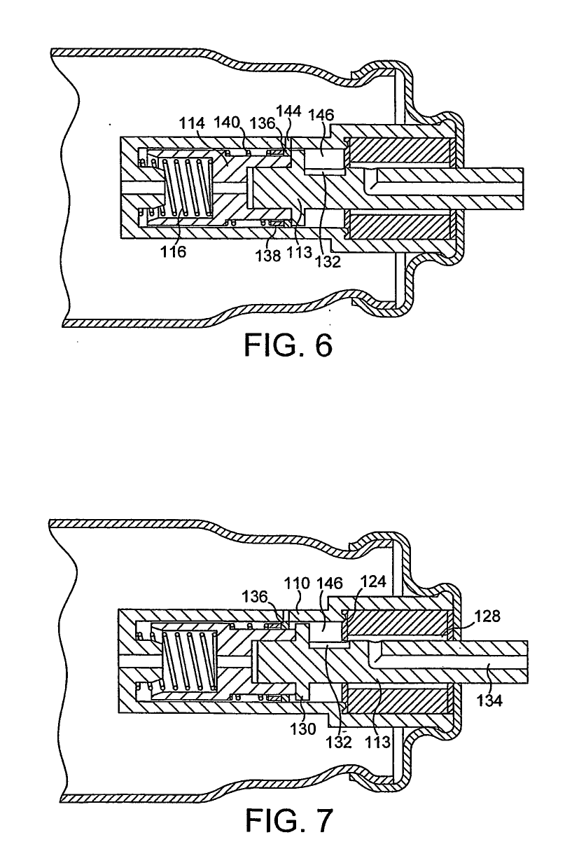

FIGS. 6 and 7 are respectively sectional views showing the valve in different states during its release;

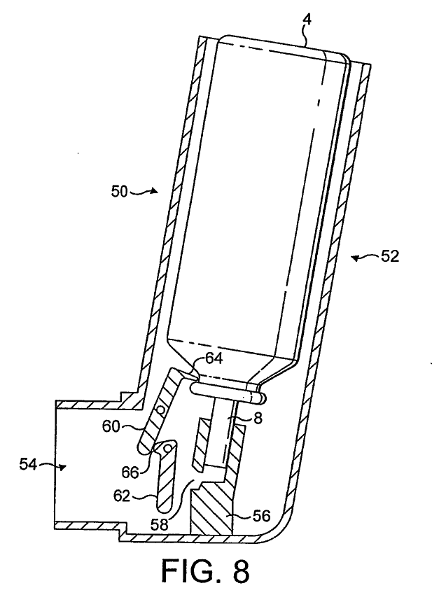

FIG. 8 is a sectional view through an inhaler in accordance with a further aspect of the invention;

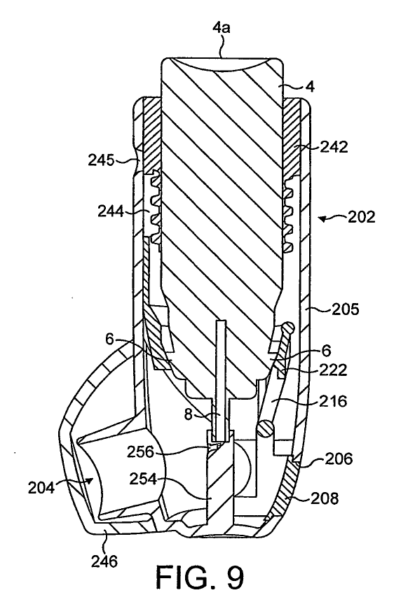

FIG. 9 is a sectional view through an inhaler of another embodiment of the invention;

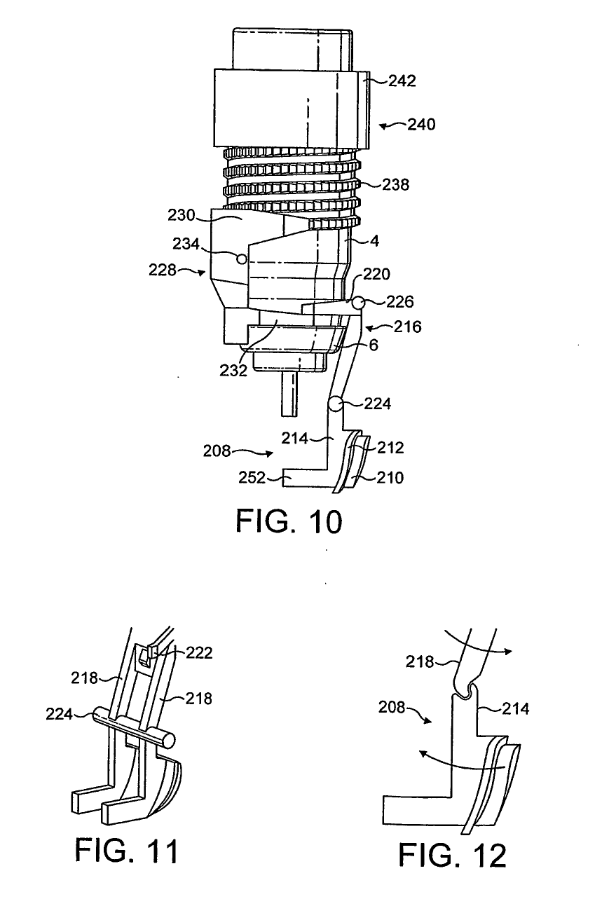

FIG. 10 is a side elevation of certain components of the inhaler of FIG. 9;

FIG. 11 is a perspective view of the flap and part of the trigger of the inhaler latch mechanism;

FIG. 12 is a sectional view through the flap and trigger shown in FIG. 11;

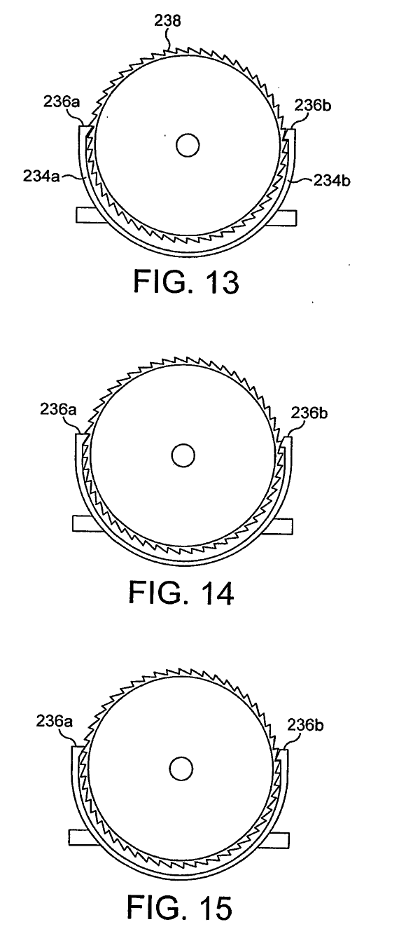

FIGS. 13 to 15 are sectional views of the escapement dose counting mechanism of the inhaler showing respectively different phases of its operation;

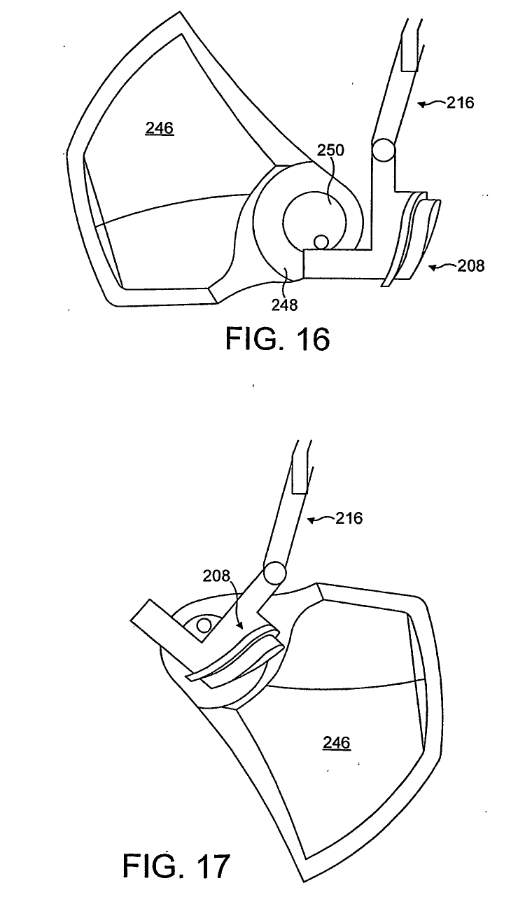

FIG. 16 is a sectional view of the mouthpiece cover and flap in the storage state; and

FIG. 17 is a view similar to FIG. 16 showing the cover in a state ready for use.

Turning to FIG. 1, there may be seen a valve arrangement 2 provided at one end of a sealed canister 4. The valve mechanism 2 is retained in the end of the canister 4 by a sealing cap 6 as is well known in the art. The valve mechanism 2 has a hollow nozzle 8 extending along the axis of the canister 4 and through an aperture in the sealing cap 6.

The housing of the valve mechanism is generally bell-shaped with a wide base flange 10a abutting the under-side of the sealing cap, a main body section lob and a narrower end neck portion 10c. The shape of the canister in the region of the sealing cap 6 is such that when the cap 6 is applied, the base flange 10a of the valve mechanism is clamped between the body of the canister 4 and the underside of the cap 6. A washer seal 12 forms a pressure-tight seal around the aperture in the cap 6 for the nozzle 8.

Turning now to FIG. 2 in which the valve mechanism may be seen in more detail, it will be seen that the nozzle member 8 is a sliding fit inside the narrowed end neck portion 10c of the valve and also in the main body portion lob as a result of a radially extending flange 14 provided part-way along the nozzle member 8.

The innermost end of the nozzle member 8 is formed with a narrow tapered head 16 defining a shoulder 18 where it joins the rest of the nozzle member 8. A compression coil spring 20 is disposed between the shoulder 18 of the nozzle member and the inner end of the neck portion 10c of the valve so as to encircle the tapered head 16. The spring 20 acts to bias the nozzle member 8 towards the front end of the valve mechanism 2 so that its radial flange 14 abuts against the washer seal 12.

Two further washer seals 22, 24 are provided around the nozzle member 8 within the main body 10b of the valve to seal against the outside of the nozzle member 8 and the inside of the valve casing 10b respectively. One of the seals 22 abuts against the inside of the shoulder formed between the main body 10b and the narrowed end neck portion 10c of the valve. The second seal 24 is spaced axially from the first. The two seals 22,24 are fixed in their axial positions by a pair of L-section spacers 26,28 which are themselves a tight interference fit in the main section lob of the valve body. The two seals 22,24 define between them a metering chamber 30 of precise predetermined volume having the shape of a rectangular-section toroid. The metering chamber 30 is in fluid communication with the axial bore 32 of the nozzle member 8 through a radial bore section 34.

On the other side of the foremost seal 24 a larger chamber 36 is defined. An aperture 38 through the wall of the main valve body 10b is provided so that the chamber 36 is in fluid communication with the interior of the canister 4.

A notch 40 is cut out of the part of the nozzle member 8 which is disposed in the larger chamber 36 in the configuration shown in FIG. 2.

Operation of the valve will now be described with reference to FIGS. 1-3. The normal rest state of the valve mechanism is shown in FIGS. 1 and 2. The canister 4 is filled with a mixture of pressurised propellant and active ingredient. The aperture 38 in the body lob of the valve means that the propellant/drug mix fills the larger fore-chamber 36 of the valve. The metering chamber 30 on the other hand is empty and at atmospheric pressure since it is open to the atmosphere through the bores 32, 34 of the nozzle member 8.

When it is desired to dispense a dose of drug from the canister, the nozzle 8 is depressed into the canister 4 against the force of the coil spring 20. This is shown in FIG. 3. In the fully depressed condition, the tip of the tapered head 16 at the end of the nozzle member 8 abuts against the end wall of the valve neck portion 10c. In this position, the nozzle 8 is moved sufficiently far into the valve that the notch 40 in the side of the nozzle member 8 is aligned with the foremost seal 24 which defines one side of the metering chamber 30. This allows the pressurised propellant/drug mix to bypass the seal 24 to enter and fill the metering chamber 30. The volume of the metering chamber 30 is precisely predetermined to isolate the required dose. It will of course be appreciated that in the depressed condition, the metering chamber 30 is closed to the atmosphere since the radial bore 34 of the nozzle member is no longer in alignment with it.

When pressure on the nozzle member 8 is released, the spring 20 returns it to its original position as shown in FIGS. 1 and 2. During the first part of this movement, the notch 40 is moved out from under the seal 24 in order to reseal the metering chamber 30. Thereafter, the radial bore 34 in the nozzle member 8 is once again brought into alignment with the metering chamber 30 thus opening the metering chamber 30 to the atmosphere. Since the pressure of the propellant in the metering chamber 30 is significantly elevated above atmospheric pressure, this will cause the propellant/drug mix to be sprayed from the end of the nozzle 8 as is well known in the art.

Thus, it will be appreciated by those skilled in the art that during a single actuation cycle of depressing and subsequently releasing the nozzle 8, a dose of propellant and drug is isolated in the metering chamber 30 and the same dose is then dispensed. This means that the canister 4 may be shaken prior to actuation to achieve a homogenous mix of drug and propellant throughout, from which a dose of the correct concentration can be isolated. Furthermore, since the nozzle would normally be released very shortly after it is depressed, there is insufficient time for the active ingredient to migrate out of the metering chamber 30.

Moreover, in the fully depressed condition shown in FIG. 3, although a dose is isolated in the metering chamber 30, the bypass notch 40 under the seal 24 means that the chamber 30 is not sealed against the interior of the canister 4. Thus, even if the nozzle were to remain in its depressed condition for a relatively prolonged period of time, migration of the active ingredient is unlikely to be a significant problem. Indeed, the contents of the metering chamber is in fact only completely sealed for a fraction of a second during the release stroke between the time when the notch 40 and the radial bore 34 of the nozzle are respectively aligned with the metering chamber 34. It will be appreciated from this that it is not necessary to waste a dose from the canister even if it has not been used for a long time.

FIG. 4 shows a cross section through a valve and part of a canister in accordance with another embodiment of the invention. As in the previous embodiment, the canister comprises a canister wall 102 closed by a cap 104 so as to define a canister interior 106 which is filled with a pressurised mixture of medicament and propellant.

The valve 108 generally comprises a valve casing 110 and a valve stem 112 mounted for axial sliding movement within it. The valve stem 112 engages at its inner end a valve stem base member 114. These two parts together form a nozzle member or plunger 113. This two-part construction of the valve plunger assists the manufacture and assembly of the valve but it is not essential—the stem 112 and base member 114 could be formed as a single integral moulding.

The base member 114 is acted upon by a main spring 116. The main spring 116 is a coil spring and is located over a boss 118 formed at the innermost end of the valve casing 110. The boss 118 has a central bore through it so that the inner part of the valve 108 is at the same pressure as the main interior of the canister 106. The pressure differential between the interior of the canister 106 and the atmosphere; and the force of the main spring 116, both act to bias the plunger 113 outwardly—i.e. towards the right as viewed from FIG. 4, into the rest state of the valve in which the valve stem 112 protrudes by the maximum amount from the cap 104 of the canister.

The valve casing 110 has a enlarged-section portion 110a at the front portion. A relatively thick annular spacer 122 is fitted into the enlarged-section portion of the casing 110a with annular seals 124, 126 being provided at either end. The annular seals seal onto the valve stem 112. The annular gap between the spacer 122 and the valve stem 112 defines a transfer chamber 128 which is delimited axially by the two annular seals 124, 126.

The valve stem 112 has a circumferential flange 130 which in the rest position shown in FIG. 4 abuts against the inner annular seal 124 to delimit the sliding movement of the plunger 113. The radius of the flange is a little shorter than that of the valve casing 110 so that it does not form a sealing fit inside the valve casing 110.

A recess is provided in the radially outer surface of the valve stem forward of the flange 130 to form a transfer port 132. Forward of the transfer port 132 is a radial port communicating with an axial bore that extends to the foremost end of the valve stem 112 and forms an outlet port 134.

Rearwardly of the valve stem flange 130 is a square-section annular sliding seal 136. An annular collar 138 behind. the sliding seal 136 transmits the force of a seal spring 140 to the seal. The other end of the seal spring 140 bears on an annular shoulder 142 formed in the valve stem base member 114. The seal spring 140 therefore biases the sliding seal 136 against the valve stem flange 130.

An aperture in the valve casing 110 in the region of the sliding seal 136 forms an inlet port 144 for the valve communicating it with the interior of the canister 106.

Operation of the valve will now be described with reference to FIGS. 5 to 7. As stated above, the rest state of the of the valve is shown in FIG. 4. The nozzle member plunger 113 is pressed into the canister to prime it. This is shown in FIG. 5. The plunger 113 must be pressed in with sufficient force to overcome the force of the main spring 116 and the pressure of the interior of the canister 116.

The movement of the plunger 113 and particularly the annular flange 130 also drives the sliding seal 136 inwardly until it passes the inlet port 144. At this point the pressurised mix of medicament and propellant in the main body of the canister 106 can enter the metering chamber 146 which has been formed in the axial space between the sliding seal 136 and the rearmost annular seal 124.

It will be seen that the transfer port 132 is now completely within the metering chamber 146. This means that the annular seal 124 seals against the outer surface of the valve stem 112 and therefore that the metering chamber 146 is sealed from the atmosphere.

It will also be appreciated that since the metering chamber 146 is at the same pressure as the interior of the canister 106, the hydraulic pressure on the plunger 113 is equalised and so the only net force acting on the plunger from within the canister is the restoring force of the main spring. Thus whilst a relatively higher force is required to prime the valve initially, thereby helping to prevent inadvertent operation, the force required to hold the plunger 113 in the primed position is relatively lower. This translates to more sensitive breath-actuation mechanism being possible.

When the external force on the plunger is removed—e.g. by releasing a latch as will. be described hereinbelow—the main spring 116 begins to drive the plunger 113 forwards again as may be seen in FIG. 6. The sliding seal is driven forward by the valve stem base member 114 acting through the seal spring 140 and seal collar 138.

FIG. 6 shows the sliding seal 136 having just passed the inlet port 144. At this point the metering chamber 146 is sealed closed since the transfer port 132 remains fully within it. The volume of the metering chamber 146 at this point thus fixes the dose which will be dispensed and so is precisely predetermined.

The main spring 116 continues to drive the plunger 113 forwards. However since the contents of the metering chamber are essentially incompressible, the sliding seal 136 is prevented from moving further forwards. The plunger 113 thus slides forward relative to the sliding seal 136 which remains stationary. This is shown in FIG. 7. It will be appreciated that this ‘separation’ between the sliding seal 136 and the plunger 113 is made possible by their ability to slide relative to one another and prevents potential problems with hydraulic lock.

In the position shown in FIG. 7, the transfer port 132 is just about to pass under the annular seal 124. Clearly further forward movement will causes this to happen, in which case the metering chamber 146 is vented to the atmosphere via the transfer chamber 128 and the outlet port 134 and the metered dose of medicament is thereby dispensed. The main spring 116 and now once again the internal pressure of the canister, combine to drive the plunger 113 further forward until the flange 130 once again abuts the annular seal 124.

The release of pressure in the metering chamber 146 also allows the sliding seal 136 to be driven forward again by the seal spring 140 which is compressed between the states in FIGS. 6 and 7. As the sliding seal 136 is driven forward, the volume of the metering chamber 146 is reduced until the sliding spring 136 returns to its rest position too as shown in FIG. 4 and in which the volume is reduced essentially to zero (a very tiny annular space between the flange 130 and the valve casing 110 being all that remains). This reduction of the volume of the metering chamber substantially to zero ensures that all of the dose is fully delivered and means that the metering chamber is not open to the atmosphere during storage.

FIG. 8 shows schematically a cross-section through an inhaler in accordance with a further aspect of the invention. The inhaler 50 comprises generally an approximately vertical canister holster portion 52 and a horizontal mouth-piece portion 54. The holster portion 52 receives the canister 4 described above with reference to FIGS. 1-3, or 4 to 7 although any canister in accordance with the principles set out herein may be used.

The nozzle 8 of the canister is received in a seat member 56 having a flared outlet 58 from which the pressurised propellant and drug mixture will be sprayed into the mouth-piece 54 when dispensed from the canister 4.

The novel feature of the inhaler is a latch mechanism comprising a pivotally mounted latch arm 60 and a hinged flap 62. The latch arm 60 is pivoted approximately half way along its length and has a pointed nose 64 at one end. The flap 62 is hinged about its upper edge. The upper edge is formed as a rounded cam surface 66.

In use, the nozzle 8 extends out of the canister 4 by its maximum amount so that the cap 6 of the canister is located above the pointed nose 64 of the latch arm 60 (not shown). When the user wishes to dispense and inhale a dose of drug from the canister, he or she first depresses the top of the canister 4 downwardly relatively to the inhaler 50. This causes the nozzle 8 to be depressed into the canister 4. As was explained above with reference to FIGS. 1 to 7, this does not cause a dose to be dispensed from the canister but does isolate a dose ready for dispensing. It is not therefore required to coordinate this action with any breathing.

As the body of the canister 4 moves downwardly, the sealing cap 6 is forced past the pointed nose 64 on the latch arm 60 which is held against the canister by the cam surface 66 bearing onto its opposite end. The nose 64 is thus hooked over the cap 6 and retains the canister 4 in its depressed condition. This is the condition shown in FIG. 8 The inhaler is now primed for dispensing the dose.

When the user is ready, he or she may then place his or her lips around the outside of the mouth-piece 54 and inhale. The subsequent movement of air through the inhaler 50 causes the flap 62 to rotate upwardly in a clockwise direction (as viewed from FIG. 8). The resulting movement of the cam surface 66 at the top of the flap 62 releases the latch arm 60 and so allows the pointed nose 64 to disengage from the cap 6. This causes the canister to return to its original position under the force stored in the spring of its valve. As will be appreciated from the description above, this causes a dose of drug and propellant to be dispensed from the canister's nozzle 8 and sprayed from the outlet 58 into the mouth-piece 54, therefore allowing it to be inhaled into the user's lungs. Thus, it will be appreciated that the user does not need to coordinate any action with his or her in-breath since the inhalation automatically causes the dose to be dispensed. The latch mechanism may be as simple as shown since only a relatively small force is required to disengage the latch and therefore release the previously stored energy from the canister valve. This small release force can easily be provided by the user's in-breath.

A further embodiment of an inhaler device in accordance with the present invention will now be described with reference to FIGS. 9-17. Turning firstly to FIG. 9, there may be seen a cross-section through the inhaler in which a pressurised medicament canister 4 has been loaded. The canister and valve thereof is preferably as described above with reference to FIGS. 4 to 7, but could equally be as described with reference to FIGS. 1 to 3 or indeed any canister having a ‘reverse’ actuation (i.e. one that dispenses on release rather than on compression) which has an appropriate external shape.

As in the previous embodiment, the inhaler generally comprises a canister holster portion 202 and a mouth-piece portion 204. In this embodiment an air inlet aperture 206 is provided in the rear wall of the inhaler opposite the mouth-piece 204. The air inlet aperture 206 is closed by a flap member 208. As may be seen more clearly in FIGS. 10 and 12, the flap member 208 comprises a plug portion 210 surrounded by a rim 212 which engages with an inset ledge around the wall of the aperture 206 to form a sealing engagement in which the outer face of the plug portion 210 is flush with the rear wall of the canister 205. The flap 208 also comprises an upwardly extending arm 214 which pivotally engages with a trigger member 216. The actual engagement between the flap 208 and the trigger 216 is somewhat similar to a knee joint and is shown more clearly in FIG. 12.

The trigger member 216 is approximately L-shaped in profile and comprises two downwardly extending legs 218 which engage with corresponding arms 214 of the flap. The upper part of the trigger member 216 is in the form of a yoke with two arms 220 extending around either side of the canister 4. The trigger member 216 also comprises a protruding detent 222. The flap 208 and trigger 216 are each pivotally mounted to the body of the inhaler by respective pivots 224, 226 so that they may rotate around mutually parallel axes which are generally perpendicular to the axis of the canister 4. This may be seen most clearly in FIG. 10.

On the diametrically opposite side of the canister 4 to the trigger member 216 is a double-ended yoke member 228. The yoke member 228 comprises upper and lower pairs of yoke arms 230, 232 respectively which also extend approximately half way round the canister 4, but from the other side to the trigger member 216. As will be seen from FIG. 10, the respective lengths of the lower arms 232 of the yoke member 228 and the upper arms 220 of the trigger member 216 are such that they overlap one another by a small amount with the trigger yoke arms 220 being on top of the lower yoke member arms 232. The yoke member 228 is also pivotally mounted to the body of the canister by a pivot 234 so that it may rock about an axis generally parallel to the pivot axes 224, 226 of the flap and trigger members respectively.

The upper yoke arms 230 each have at their distal ends an inwardly projecting pawl 236 which may engage with a helical saw-tooth track 238 provided around the circumference of a counter member 240. The counter member 240 is in the general form of a cylindrical sleeve having the helical saw-tooth track 238 around the lower part and a display collar 242 around the upper part. Although not shown in the diagrams, the upper collar 242 has marked on it a series of numbers arranged in a helix of the same length and pitch of the saw-tooth track 238. As can be seen from FIG. 9, the inside wall of the canister holster 202 is threaded in the region of its upper portion 244 to engage with the thread formed by the helical saw-tooth track 238 on the counter member 240.

A window 245 is formed in the front wall of the inhaler to allow one of the marked figures on the collar 242 to be viewed from outside the inhaler.

Moving to the exterior of the inhaler, a hinged mouth-piece cover 246 is provided to cover the mouth-piece 204. As may be seen from FIG. 16, the mouth-piece cover 246 comprises a shaped protrusion 248 from its pivot boss 250. When the mouth-piece cover 246 is in the storage position shown in FIGS. 9 and 16, the pivot boss protrusion 248 engages with a horizontally extending arm 252 of the flap member 208 to lock the flap member into place. However, when the mouth-piece cover 246 is rotated away from the mouth-piece 204 the pivot boss protrusion 248 disengages the flap member 208 to allow it to pivot as is shown in FIG. 17.

Operation of the inhaler shown in FIGS. 9 to 17 will now be described. A pressurised medicament canister 4 is loaded into the inhaler so that its valve stem 8 is received in a valve seat 254 so that the valve stem is in fluid communication with a spray vent opening 256, as in the previous embodiment. The storage position of the inhaler and loaded canister is shown in FIG. 9. When it is desired to dispense a dose of medicament, pressure is applied to the base 4a of the canister in order to press the valve stem 8 into the body of the canister. This primes the metering chamber of the canister valve (not shown) with a dose of the pressurised medicament and propellant mixture. The downward movement of the canister body causes the cap rim 6 thereof to pass and clip under the detent 222 of the trigger member 216. This latches the canister in its primed position.

The user then hinges the mouth-piece cover 246 away from the mouth-piece 204 through approximately 180° so that it forms a guard over the air inlet 206 at the rear of the inhaler. As may be seen by comparing FIGS. 16 and 17, rotating the mouth-piece cover 246 from the closed to the open position disengages the mounting boss protrusion 248 thereof from the flap member 208.

The user then places his or her mouth around the mouth-piece 204 and takes in a deep breath. The interacting threads 244, 238 on the canister holster and the counter-member respectively form a reasonably air tight seal and thus when the user begins to breath in, the interior of the inhaler undergoes a sudden drop in pressure. This pressure differential across the flap 208 causes it to hinge into the inhaler in a clockwise direction as may be seen more clearly in FIG. 12. The inwardly pivoting movement of the flap member 208 allows the trigger member 216 to rotate in the opposite direction as the upper arm 214 of the flap member disengages from the lower legs 218 of the trigger member (see FIG. 12).

As the trigger member 216 pivots in an anti-clockwise sense as seen from FIG. 9, the detent 222 thereon is allowed to disengage from the rim 6 of the canister to release the canister 4 and allow it to travel upwardly as the valve stem 8 is pushed out therefrom by the canister's internal spring (not shown). This causes a metered dose of the medicament to be dispensed from the canister 4 through the valve stem 8 and out of the spray outlet 256 into the mouth-piece 204 to be entrained into the user's in-breath.

It will thus be appreciated that the mechanism described allows a metered dose of medicament to be co-ordinated with the in-breath of the user by virtue of the dispensation being triggered by the user's breath. As has been discussed previously, this significantly increases the ease of use of such devices and also permits greater consistency in the actual dose received by the user. It will further be appreciated that the engagement between the mouth-piece cover 246 and the flap member 208 ensures that the mechanism will not be accidentally activated until the user is ready to use the device by opening the mouth-piece cover.

The mechanism for counting the number of doses dispensed from a particular canister will now be described with particular reference to FIGS. 10, 13, 14 and 15. The rest position of the dose counting mechanism is represented in FIG. 13. This Figure shows a sectional view from above of the two upper yoke arms 234a, 234b, of the yoke member 228 and a portion of the helical saw-tooth track 238. As may be seen by considering FIG. 13 in more detail, in the rest position, the left and right hand pawls 236a, 236b are engaged with respective teeth of the track 238 on diametrically opposed sides thereof.

As will be appreciated from FIG. 10, when the user presses down the canister 4 in order to prime it, the tapering profile of the cap rim 6 on the lower legs 218 of the trigger member acts as a cam to rotate the trigger member 216 through a small arc in an anti-clockwise direction as viewed from FIG. 10 which in turn causes the upper arms 220 of the trigger member to press downwardly on the lower arms 232 of the yoke member. This causes the yoke member 228 to rock forwardly as shown by the direction of the arrow in FIG. 13. The effect of this is shown in FIG. 14. It will be seen that the left hand pawl 236a drives the counter member 240 round by half a tooth pitch. When this half pitch rotation is complete, the mechanism again looks like that shown in FIG. 14 as the right hand pawl 236b engages over the next counter tooth.

When the breath actuator mechanism is released as described above, the upward movement of the cap rim 6 acts on the lower yoke arms 232 of the yoke member to cause it to rock back again. The effect of this is shown in FIG. 15. In this case, the right hand pawl 236b causes the counter member 240 to rotate by another half a tooth pitch. Again, when the rotation is completed the counter is at rest in the position shown in FIG. 13. However, it will be appreciated that the counter member 240 will have been driven round by one counting increment. The effect of this is that the number marked on the upper collar 242 which is visible through the window 245 will increase by one. By this mechanism, the number of doses dispensed may be counted. Of course, the numbers may be printed in reverse on the collar so that an estimate of the remaining number of doses is given rather than the actual number used.

Since the toothed track 238 is helical and cooperates with the thread 244 on the inside of the canister holster, as well as rotating, the counter member also moves gradually downwardly with respect to the inhaler body as it rotates. This means that when the end of the count is reached and therefore the canister 4 is replaced, the counting mechanism must be rest by turning it in reverse so that the counter member again rises in the inhaler.

It will be appreciated by those skilled in the art that the embodiments described above are only specific examples of how the principles of the invention may be implemented and there are many possible variants within the scope of the invention.

1. A metered dose inhaler comprising means for receiving a pressurised medicament canister; and a breath-actuated latch mechanism arranged in use to latch said canister in a depressed condition and further to release said latch in response to inhalation through the inhaler by a user, thereby releasing said canister from a depressed condition to dispense a metered dose.

2. An inhaler as claimed in claim 1 wherein said latch mechanism comprises a pivotally mounted latch arm operatively associated with a hinged flap arranged to rotate upon inhalation by a user.

3. An inhaler as claimed in claim 2 wherein said hinged flap is provided so as to close an air inlet to the inhaler.

4. An inhaler as claimed in claim 2 comprising means for positively restoring said flap to its rest position.

5. An inhaler as claimed in claim 4 wherein said flap is at least partially restored to said rest position by re-priming said latch mechanism.

6. An inhaler as claimed in claim 4 comprising an externally-operated actuator for restoring said flap.

7. An inhaler as claimed in claim 6 wherein said actuator comprises or is operated by a cover for the mouthpiece of the inhaler which is arranged to restore or to help to restore the flap when the cover is closed over the mouthpiece.

8. An inhaler as claimed in claim 6 said external actuator is arranged to apply a sealing force on the flap.

9. A breath-actuated inhaler comprising a mouthpiece, a mouthpiece cover and an air inlet, the mouthpiece cover being arranged such that as it is brought over the mouthpiece it acts on a flap to hold the flap in a position where it closes the air inlet.

10. An inhaler as claimed in claim 9 wherein said cover acts to provide a sealing force on the flap.

11. An inhaler as claimed in of claims 7 wherein the mouthpiece cover is arranged to form a guard over the air inlet to prevent inadvertent blockage of the air inlet during inhalation.

12. An inhaler comprising a mouthpiece, a mouthpiece cover and an air inlet wherein the mouthpiece cover is movable from a first position in which it covers said mouthpiece to a second position in which it forms a guard over said air inlet to prevent blockage thereof in use.

13. An inhaler as claimed in claim 11 wherein said mouthpiece cover is pivotally mounted.

14. An inhaler as claimed in claim 1 adapted so that when in use a canister is inserted into the inhaler, the interior of the inhaler is substantially closed except for a mouthpiece and an air inlet.

15. An inhaler as claimed in claim 1 comprising a dose counter for counting the number of doses dispensed from said canister said dose counter comprising a counter member having a toothed track arranged substantially in a helix and means for incrementally advancing said counter member via said toothed track for each time a dose is dispensed from said canister.

16. An inhaler as claimed in claim 15 wherein said dose counter is operatively associated with said latch mechanism.

17. A metered dose inhaler for receiving a pressurised medicament canister and comprising a dose counter for counting the number of doses dispensed from said canister said dose counter comprising a counter member having a toothed track arranged substantially in a helix and means for incrementally advancing said counter member via said toothed track for each time a dose is dispensed from said canister.

18. An inhaler as claimed in claim 16 comprising an escapement mechanism in which a reciprocating motion from depressing and releasing the canister is translated into an incremental rotary motion of the counter member.

19. An inhaler as claimed in claim 18 wherein the escapement mechanism comprises an escapement yoke comprising a pair of pawls which are arranged to engage with teeth on opposite sides of the toothed track when the canister is respectively depressed and released.

20. An inhaler as claimed in any of claims 17 wherein said dose counter is operatively associated with a canister latch mechanism.

21. A pressurised canister for dispensing a metered dose of fluid therefrom having a valve comprising a sliding nozzle member biased towards a rest position but moveable against said bias to a priming position in which a metering chamber is defined within the valve such that when said nozzle member is released a metered dose is dispensed, the valve further comprising a sliding seal delimiting said metering chamber and slidable relative to the nozzle member, said sliding seal being biased in use to reduce the volume of the metering chamber substantially to zero once the metering chamber has been vented to the atmosphere via the nozzle member.

22. A canister as claimed in claim 21 wherein the sliding seal is exposed to the pressure of the contents of the canister in order to apply at least some of the force required to move the seal.

23. A canister as claimed in claim 22 further comprising a spring within the valve to act on the sliding seal.

24. A canister as claimed in claim 23 wherein said spring is arranged to act between the nozzle member and the sliding seal to give a biasing force on the sliding seal relative to the nozzle member.

25. A canister as claimed in claim 23 comprising an intermediate collar between said spring and said seal.

26-32. (canceled)

33. A valve for a canister said valve comprising a metering chamber, an inlet for fluidly communicating with the interior of a canister and a hollow nozzle resiliently biased into a first position in which the nozzle is in fluid communication with the metering chamber, but moveable against said resilient bias into a second position in which the inlet is in fluid communication with the metering chamber.

34. (canceled)

35. An inhaler device comprising means for latching a canister in its depressed condition and means for releasing said latch upon inhalation by a user, thereby releasing said canister from its depressed condition.

36. A pressurised canister for a metered dose inhaler comprising a resiliently biased nozzle and arranged to dispense a metered dose of fluid from said nozzle upon releasing the nozzle from its depressed condition, wherein said canister comprises a valve including a metering chamber and a hollow nozzle resiliently biased into a first position in which said nozzle is in fluid communication with the metering chamber, said nozzle being moveable against said resilient bias to a second position in which the metering chamber is in fluid communication with the interior of the canister.
